(19) United States
(12) Patent Application Publication
(10) Pub. No.: US 2007/0225512 A1
(43) Pub. Date: Sep. 27, 2007

(54) SYNTHESIS OF A MIXTURE OF SULFATED ESTROGENS USING A SULFUR TRIOXIDE COMPLEX

(76) Inventor: Thomas W. Leonard, Wilmington, NC (US)

Correspondence Address: BUCHANAN, INGERSOLL & ROONEY PC, POST OFFICE BOX 1404, ALEXANDRIA, VA, 22313-1404, US

(21) Appl. No.: 11/757,764

(22) Filed: Jun. 4, 2007

Related U.S. Application Data
(63) Continuation of application No. 11/259,407, filed on Oct. 26, 2005, now abandoned.
(63) Continuation of application No. 10/041,916, filed on Jan. 8, 2002, now abandoned.

(51) Int. Cl. C07J 1/00

(52) U.S. Cl. 552/626; 552/625

(57) ABSTRACT

A process of producing a stable composition comprising a mixture of sulfated estrogens. The mixture of sulfated estrogens may comprise sulfated alkali metal salts of Δ^{8,9}-dehydroestrone, estrone, and derivatives thereof or other estrogens. The process comprises the steps of sulfating such mixture of alkali metal salts of Δ^{8,9}-dehydroestrone, estrone, and derivatives thereof with a sulfur trioxide complex and adding tris(hydroxymethyl)aminomethane.

### CROSS REFERENCE TO RELATED APPLICATION

The instant application is a continuation application of U.S. Continuation application Ser. No. 11/259,407, filed Oct. 26, 2005 and claims priority to U.S. application Ser. No. 10/041,916, filed Jan. 8, 2002, the disclosures of which are incorporated herein by reference in their entireties.

### FIELD OF THE INVENTION

The present invention relates to a process for the synthesis of a mixture of sulfated estrogens which may comprise sulfated Δ^{8,9}-dehydroestrone, estrone, equilin, and derivatives thereof, among others.

### BACKGROUND

Naturally occurring estrogenic compositions are used in medical treatments to alleviate the symptoms of menopausal syndrome and osteoporosis/osteopenia in estrogen deficient women, prevent cardiovascular disease in men and women, and treat other hormone related disorders. The estrogenic components of the naturally occurring estrogenic compositions include sulfate esters of estrone, as disclosed in U.S. Pat. No. 2,834,712.

The synthesis of sulfated estrogens has been described in past publications. For example, U.S. Pat. No. 5,288,717 to Raveendranath et al. (Alkali Metal 8,9-Dehydroestrone Sulfate Esters) teaches a process of synthesizing alkali metal salts of 8,9-dehydroestrone (Δ^{8,9}-DHE) and its sulfate ester free from other conjugated esters present in material found in natural sources of mixed esters. In the process of Raveendranath, an alkali metal salt of Δ^{8,9}-DHE is initially produced followed by sulfation with sulfur trioxide-trimethylamine complex (SO₃-TMA) under mild conditions in an apolar, aprotic solvent such as tetrahydrofuran (THF) with simultaneous or subsequent addition of tris(hydroxymethyl)aminomethane (TRIS) as a stabilizer. The alkaline bases employed in the production of the initial intermediates of Δ^{8,9}-DHE are preferably sodium or potassium in the form of their hydrides and lithium as n-butyl lithium. This process provides a product free of other conjugated esters and does not teach production of several compounds at once.

U.S. Pat. No. 5,998,639 to Raijmakers, et al. (Sulfatation of Estrogen Mixtures) teaches a process for the preparation of a mixture of sulfated estrogens containing Δ^{8,9}-DHE or derivatives thereof. In the process of Raijmakers, an estrogen mixture is obtained by isomerization of equilin or a derivative thereof using lithium salts of ethylene diamine. This mixture is sulfated with sulfuric acid/acetic anhydride/pyridine. The mixture of crude pyridinesulfates is treated with sodium hydroxide in methanol, yielding a mixture in a specific ratio of Δ^{8,9}-DHE sodium sulfate and one or more of, for example, equilin sodium sulfate, 17α-dihydro equilin sodium sulfate, 17β-dihydro equilin sodium sulfate, 17α-estradiol sodium sulfate, and 17β-estradiol sodium sulfate.

There remains a need for an efficient process of producing a stable composition of a mixture of sulfated estrogens.

### SUMMARY OF THE INVENTION

The present invention provides processes for the production of stable compositions comprising complex mixtures of sulfated estrogens. Previous synthetic procedures have involved synthesis of an estrogen, or a mixture of estrogens, from synthesis of a precursor. The present invention provides for the synthesis of complex estrogens by parallel synthetic processes on a mixture of precursors.

In one aspect, the estrogens can comprise at least two of Δ^{8,9}-DHE, estrone, equilin, or derivatives thereof. The mixture of sulfated estrogens would correspondingly comprise sulfated alkali metal salts of Δ^{8,9}-DHE, estrone, equilin, or derivatives thereof. These compounds are obtained in ratios not obtained when synthesized individually. The process comprises reacting a sulfur trioxide complex with a mixture of alkali metal salts of estrogens; adding a stabilizing amount of TRIS; and recovering the stable composition comprising the mixture of sulfated estrogens and TRIS. The process may further comprise reacting a mixture of estrogens with an alkali metal hydride to provide the mixture of alkali metal salts of estrogens. The process may be performed in an apolar, aprotic solvent. All steps of the process may also be performed in a single reaction vessel.

One advantage of the present invention is that the process produces a mixture of sulfated estrogens in a single vessel. In this respect, the mixture of alkali metal salts of Δ^{8,9}-DHE, estrone, equilin, and/or related substances is sulfated simultaneously to provide a complex mixture of sulfated estrogen alkali metal salts having a potentially altered estrogenic composition and with ratios of the three primary estrogens that would not have been produced if synthesized individually. Furthermore, the entire reaction sequence of the process may be performed in a single vessel without isolating intermediate products.

In another aspect of the present invention, the process produces a mixture of sulfated estrogens in a specific ratio. By way of example, a mixture of estrogens comprising a specific ratio of Δ^{8,9}-DHE, estrone, equilin and derivatives thereof is provided. The process of the present invention is performed on this mixture of estrogens and produces a mixture of sulfated estrogens in the same approximate ratios as that of the starting estrogens.

### DETAILED DESCRIPTION OF THE ILLUSTRATIVE EMBODIMENTS

According to the invention, a mixture of sulfated estrogens is produced, preferably using a single vessel. In the process of the present invention, a mixture of alkali metal salts of estrogens may be prepared from a first mixture of estrogens. Typically, the first mixture will contain at least two estrogens. The estrogens may be any estrogenic compound, including Δ^{8,9}-DHE, estrone, equilin, 17α-estradiol, 17β-estradiol, 17α-dihydroequilin, 17β-dihydroequilin, equilenin, 17α-dihydroequilenin, 17β-dihydroequilenin, 17α-Δ^{8,9}-dehydroestradiol, 17β-Δ^{8,9}-dehydroestradiol, 6-OH equilenin, 6-OH 17α-dihydroequilenin, 6-OH 17β-dihydroequilenin, ethinyl estradiol, and estradiol valerate, and derivatives thereof. Derivatives thereof, as used herein includes any compounds derived from or related to the estrogenic compounds named herein.

The mixture of alkali metal salts of estrogens may be prepared by reacting the mixture of estrogens with an alkali metal hydride in an apolar, aprotic solvent. The mixture of alkali metal salts of estrogens may be sulfated using a sulfur trioxide complex in an apolar, aprotic solvent. For stability, an amount of TRIS may be added to the mixture of sulfated estrogens.

Accordingly, the general synthetic scheme as it applies to estrone and ^{Δ8,9}DHE is of the present invention is as follows:

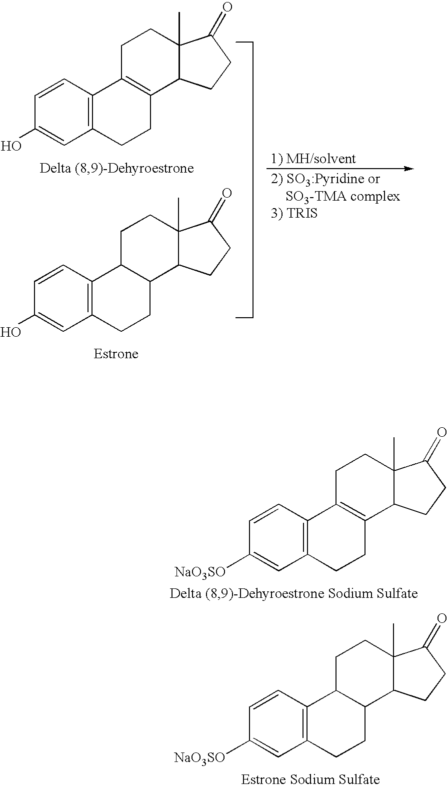

According to the general synthetic scheme of the present invention, a mixture of estrogens are reacted with an alkali metal hydride (MH), including for example, NaH, KH, LiH, and the like. This reaction may be performed in an apolar, aprotic solvent, including for example, THF, dioxane, diethyl ether, and the like. Where the mixture of estrogens comprises Δ^{8,9}-DHE, estrone, and derivatives thereof, this reaction produces a mixture of alkali metal salts of estrogens comprising alkali metal salts of Δ^{8,9}-DHE, estrone, and derivatives thereof.

The mixture of alkali metal salts of estrogens is reacted with a sulfur trioxide complex, including for example, SO₃-TMA, SO₃-pyridine, and the like. This reaction also may be performed in an apolar, aprotic solvent, including, THF, dioxane, diethyl ether, and the like. Where the mixture of estrogens comprises Δ^{8,9}-DHE, estrone, and derivatives thereof, this reaction produces a mixture of sulfated alkali metal salts of estrogens comprising alkali metal salts of Δ^{8,9}-DHE, estrone, and derivatives thereof.

To the mixture of sulfated estrogens is added a stabilizing amount of TRIS. These three reaction steps may be performed sequentially in a single reaction vessel without isolating the intermediate products. The composition comprising the mixture of sulfated estrogens and TRIS is recovered. As one of skill in the art would readily recognize, the composition may be recovered by any number of ways, including, for example, filtration, extraction, and the like. The resulting product may also be purified by any number of purification techniques, also well known in the art, including, for example, recrystallization, chromatography, and the like.

The synthetic scheme of the invention may be applied to any mixture of estrogens or their derivatives. These mixtures typically will comprise at least two estrogens or corresponding alkali metal salts of the estrogens.

### EXAMPLES

The invention will be further explained by the following illustrative examples that are intended to be non-limiting.

### Example 1

Sodium hydride (NaH) (0.77 g, ˜0.0304 mole) and THF (60 mL) under nitrogen atmosphere were added to a dry, 500 mL, three-neck, round bottom flask equipped with an air condenser, a 100 mL addition funnel, and magnetic stir bar. The suspension was stirred and cooled to 0-5° C. Next, Δ^{8,9}-DHE (5.11 g, ˜0.0187 mole) dissolved in 75 mL THF, and solid estrone (1.27 g, ˜0.0046 mole) were added at 0-5° C. under a nitrogen atmosphere. After 30 minutes, the cooling bath was removed to allow the reaction mixture to attain ambient temperature, and the mixture was stirred for 2-2.5 hours at 20-22° C. Sulfur trioxide-pyridine complex (SO₃-Pyridine) (4.05 g, ˜0.025 mole) was added in small batches to the reaction mixture. After stirring for 30 minutes, TRIS (2.82 g, ˜0.0233 mole) was added, and stirring was continued overnight at 20-22° C. under a nitrogen atmosphere. The mixture was transferred into a 1 L round bottom flask. The solvent was evaporated under high vacuum (0.15 mm of Hg pressure) at 20° C., and then the pyridine was removed at 29-30° C. This step was repeated by adding 20 mL of fresh THF to the residue. The residue was taken up in 130 mL of deionized water, and the aqueous solution extracted nine times with 50 mL of diethyl ether to remove unreacted Δ^{8,9}-DHE and estrone. To the 130 mL of the aqueous solution were added 90 mL of deionized water and 1.6 g of activated carbon, “Darco.” This was stirred for 30 minutes and filtered through a sintered glass funnel using filter agent “Celite-521.” The carbon treatment was repeated by adding 1.6 g of “Darco,” stirring for another 30 minutes, and filtering through a sintered glass funnel using filter agent “Celite-521.” The TLC (thin-layer chromatography) of this solution [CHCl₃:MeOH:NH₄OH (25:5:1)] did not show a spot corresponding to Δ^{8,9}-DHE. The filtrate was lyophilized to obtain 9.1 g of light tan colored solid. The sample was analyzed by HPLC (high performance liquid chromatography), weight % process.

Results

| | |
|---|---|
| Total % HPLC wt. % Assay (DHES + ES) = | 55.0 |
| Molar Ratio of DHES:ES = | 1:0.29 |
| Molar Ratio of (DHES + ES):TRIS = | 1:1.73 |
| Moisture content by Karl-Fisher = | 3.2% |

### Example 2

The general process described in Example 1 was followed, except that SO₃-Pyridine was replaced by SO₃-TMA, and resultantly, the trimethylamine, as opposed to the pyridine, was removed.

Starting Materials and Reagents:

| Compound (purity) | Weight | Molar Amount |
|---|---|---|
| NaH (95%) | 0.64 g | ˜0.0253 mole |
| Δ^{8, 9}-DHE (98%) | 5.02 g | ˜0.0183 mole |
| Estrone (99%) | 1.22 g | ˜0.0045 mole |
| SO₃-TMA (98%) | 3.66 g | ˜0.0258 mole |
| TRIS (99.9%) | 2.82 g | ˜0.0233 mole |

Results

| | |
|---|---|
| Yield = | 8.1 g |
| HPLC Wt. % assay: DHES % = | 44.70% |
| ES % = | 9.54% |
| Total HPLC wt. % assay(DHES + ES) = | 54.24 |
| Molar Ratio of DHES:ES = | 1:0.21 |
| Molar Ratio of (DHES + ES):TRIS = | 1:1.96 |

### Example 3

The general process described in Example 2 was followed.

Starting Materials and Reagents:

| Compound (purity) | Weight | Molar Amount |
|---|---|---|
| NaH (95%) | 2.01 g | ˜0.0793 mole |
| A^{8, 9}-DHE (98%) | 10.0 g | ˜0.0366 mole |
| Estrone (99%) | 2.55 g | ˜0.0093 mole |
| SO₃-TMA (98%) | 11.5 g | ˜0.0811 mole |
| TRIS (99.9%) | 8.47 g | ˜0.0699 mole |

Results

| | |
|---|---|
| Yield | 23.79 g |
| HPLC Wt. % assay: DHES % | 31.5% |
| ES % | 9.1% |
| Total HPLC wt % assay(DHES + ES) | 40.6 |
| Molar Ratio of DHES:ES | 1:0.30 |
| Molar Ratio of (DHES + ES):TRIS | 1:2.69 |

Summary of Results

| | Molar Ratios Starting Materials of | | | | | | Results Molar Ratios | | |
|---|---|---|---|---|---|---|---|---|---|
| Exp. | DHE + E | NaH | SO₃-Amine | TRIS | DHES % | ES % | DHES:ES | (DHES + ES):TRIS | % Yield |
| 1 | 1 | 1.30 | 1.07 | 1.00 | 42.8% | 12.2 | 1:0.29 | 1:1.73 | 55.00 |
| 2 | 1 | 1.11 | 1.13 | 1.02 | 44.7% | 9.54 | 1:0.21 | 1:1.96 | 54.24 |
| 3 | 1 | 1.77 | 1.73 | 1.52 | 31.5% | 9.1 | 1:0.30 | 1:2.69 | 40.60 |

### Example 4

Chromatograms were compared to illustrate the differences between the processes taught herein and methods producing only one estrogenic compound at a time. Each chromatogram was obtained with the same chromatographic procedures. The chromatograms are detailed in Table 1 below which provides the counts per peak over time for the production of each of estrone, equilin, Δ^{8,9}-DHE (Delta-8,9), and a combination of these compounds produced by the processes of the invention (3-Combi). The table also provides ratios at each time indicated comparing each individual compound to the combination of estrogens prepared as taught herein. These relative ratios show the distinct differences in the results of the individual processes versus the combined process. As illustrated in the table, the processes of the invention enable the production of different ratios of estrogen products than can be obtained by preparing estrogenic compounds separately.

TABLE 1

| | | | | | Ratio | | |
|---|---|---|---|---|---|---|---|
| Time | Counts/Peak | | | | Estrone: | Equilin: | Delta-8, 9: |
| (min) | Estrone | Equilin | Delta-8, 9 | 3-Combi | 3-Combi | 3-Combi | 3-Combi |
| 25.721 | 0.50 | 0.50 | 0.50 | 3.05 | 0.16 | 0.16 | 0.16 |
| 24.984 | 0.50 | 0.50 | 0.50 | 6.15 | 0.08 | 0.08 | 0.08 |
| 22.152 | 0.50 | 53.81 | 0.50 | 139.62 | 0.00 | 0.39 | 0.00 |
| 21.975 | 0.50 | 0.50 | 0.50 | 32.07 | 0.02 | 0.02 | 0.02 |
| 21.634 | 0.50 | 0.50 | 0.50 | 4.33 | 0.12 | 0.12 | 0.12 |
| 20.237 | 0.50 | 0.50 | 204.47 | 41.58 | 0.01 | 0.01 | 4.92 |
| 19.436 | 0.50 | 0.50 | 10.50 | 31.66 | 0.02 | 0.02 | 0.33 |
| 18.722 | 4.08 | 0.50 | 0.50 | 31.13 | 0.13 | 0.02 | 0.02 |
| 18.376 | 0.50 | 0.50 | 15.00 | 74.69 | 0.01 | 0.01 | 0.20 |
| 15.818 | 0.50 | 15.51 | 1.00 | 3.27 | 0.15 | 4.74 | 0.31 |
| 12.591 | 0.50 | 0.50 | 3.91 | 18.93 | 0.03 | 0.03 | 0.21 |
| 11.944 | 0.50 | 0.50 | 15.75 | 2.78 | 0.18 | 0.18 | 5.67 |
| 10.981 | 0.50 | 0.50 | 0.50 | 4.22 | 0.12 | 0.12 | 0.12 |
| 10.373 | 0.50 | 0.50 | 0.50 | 2.68 | 0.19 | 0.19 | 0.19 |
| 10.222 | 0.50 | 0.50 | 0.50 | 2.18 | 0.23 | 0.23 | 0.23 |
| 9.910 | 0.50 | 0.50 | 8.57 | 3.33 | 0.15 | 0.15 | 2.58 |
| 8.901 | 0.50 | 0.50 | 0.50 | 4.01 | 0.12 | 0.12 | 0.12 |

Various modifications and alterations of this invention will become apparent to those skilled in the art without departing from the scope and spirit of this invention.

That which is claimed:

1. A process for the production of a stable composition comprising a mixture of sulfated estrogens, the process comprising the steps of:
a) reacting a sulfur trioxide complex with a mixture of at least two alkali metal salts of estrogens to provide a mixture of sulfated alkali metal salts of estrogens, wherein the estrogens are selected from the group consisting of Δ^{8,9}-dehydroestrone, estrone, equilin, 17α-estradiol, 17β-estradiol, 17α-dihydroequilin, 17β-dihydroequilin, equilenin, 17α-dihydroequilenin, 17β-dihydroequilenin, 17α-Δ^{8,9}-dehydroestradiol, 17β-Δ^{8,9}-dehydroestradiol, 6-OH equilenin, 6-OH 17α-dihydroequilenin, 6-OH 17β-dihydroequilenin, ethinyl estradiol, and estradiol valerate;
b) adding a stabilizing amount of tris(hydroxymethyl)aminomethane; and
c) recovering the stable composition comprising the mixture of sulfated estrogens and tris(hydroxymethyl)aminomethane, wherein the mixture of sulfated estrogens is produced in a specific ratio when compared to a process wherein sulfated estrogens are synthesized individually.

2. The process according to claim 1 wherein the estrogens comprise at least two of Δ^{8,9}-dehydroestrone, estrone, equilin, 17α-estradiol, 17β-estradiol, 17α-dihydroequilin, and 17β-dihydroequilin.

3. The process according to claim 1 wherein the sulfur trioxide complex is selected from the group consisting of sulfur trioxide-pyridine and sulfur trioxide-trimethylamine.

4. The process according to claim 1 wherein the alkali metal salt is selected from the group consisting of lithium, sodium, and potassium.

5. The process according to claim 1 wherein steps a) and b) are performed in an apolar, aprotic solvent.

6. The process according to claim 5 wherein the solvent is tetrahydrofuran.

7. The process according to claim 1 wherein at least steps a) and b) are performed in a single reaction vessel.

8. The process according to claim 1 wherein the mixture of sulfated estrogens are produced in a specific ratio by starting with a specific ratio of at least two estrogenic compounds selected from the group consisting of Δ^{8,9}-dehydroestrone, estrone, equilin, 17α-estradiol, 17β-estradiol, 17α-dihydroequilin, and 17β-dihydroequilin.

9. The process according to claim 1 further comprising the step of obtaining the mixture of alkali metal salts of estrogens by reacting a mixture of estrogens with an alkali metal hydride in an apolar, aprotic solvent.

10. The process according to claim 9 wherein the sulfur trioxide complex is selected from the group consisting of sulfur trioxide-pyridine and sulfur trioxide-trimethylamine.

11. The process according to claim 9 wherein the alkali metal salt is selected from the group consisting of lithium, sodium, and potassium.

12. The process according to claim 9 wherein the apolar, aprotic solvent is tetrahydrofuran.

13. The process according to claim 9 wherein at least steps a and b are performed in a single reaction vessel.

14. The process according to claim 9 wherein the sulfated estrogens are produced in a specific ratio by starting with specific ratios of estrogenic compounds selected from the group consisting of Δ^{8,9}-dehydroestrone, estrone, equilin, 17α-estradiol, 17β-estradiol, 17α-dihydroequilin, and 17β-dihydroequilin.

15. A process for the production of a stable composition comprising a mixture of sulfated estrogens, the process comprising the steps of:
a) reacting a mixture of at least two estrogens with sodium hydride in an apolar, aprotic solvent to provide a mixture of alkali metal salts of the estrogens;
b) reacting sulfur trioxide-trimethylamine with the mixture of alkali metal salts of estrogens in an apolar, aprotic solvent to provide a mixture of sulfated alkali metal salts of estrogens;
c) adding a stabilizing amount of tris(hydroxymethyl)aminomethane; and
d) recovering the stable composition comprising the mixture of sulfated estrogens and tris(hydroxymethyl)aminomethane, wherein the mixture of sulfated estrogens is produced in a specific ratio.

16. The process according to claim 15 wherein the mixture of estrogens comprises at least two estrogenic compounds selected from the group consisting of Δ^{8,9}-dehydroestrone, estrone, equilin, 17α-estradiol, 17β-estradiol, 17α-dihydroequilin, and 17β-dihydroequilin.

17. The process according to claim 15 wherein the apolar, aprotic solvent is tetrahydrofuran.

18. The process according to claim 15 wherein at least steps a, b and c are performed in a single reaction vessel.

19. The process according to claim 15 wherein the sulfated estrogens are produced in a specific ratio by starting with a specific ratio of at least two estrogenic compounds selected from the group consisting of Δ^{8,9}-dehydroestrone, estrone, equilin, 17α-estradiol, 17β-estradiol, 17α-dihydroequilin, and 17β-dihydroequilin.

20. A process for the production of a stable composition comprising a mixture of sulfated estrogens, the process comprising the steps of:
a) reacting a sulfur trioxide complex with a mixture of alkali metal salts of estrogens to provide a mixture of sulfated alkali metal salts of estrogens, wherein the estrogens are selected from the group consisting of α^{8,9}-dehydroestrone, estrone and equilin;
b) adding a stabilizing amount of tris(hydroxymethyl)aminomethane, wherein steps a) and b) are conducted in a single reaction vessel; and
c) recovering the stable composition comprising the mixture of sulfated estrogens and tris(hydroxymethyl)aminomethane, wherein the mixture of sulfated estrogens is produced in a specific ratio that is not achieved when the sulfated estrogens are synthesized individually.

21. The process according to claim 1, wherein the specific ratio of the mixture of sulfated estrogens is about the same as the ratio of the starting estrogenic compounds.
